(19) United States
(12) Patent Application Publication
(10) Pub. No.: US 2025/0082956 A1
(43) Pub. Date: Mar. 13, 2025

(54) METHODS OF ENERGIZING MERIDIANS

(71) Applicant: Tesla BioHealing, Inc., Milford, DE (US)

(72) Inventors: James Zhou Liu, Milford, DE (US); Yuehua Gu, Milford, DE (US)

(21) Appl. No.: 18/882,605

(22) Filed: Sep. 11, 2024

Related U.S. Application Data
(60) Provisional application No. 63/581,755, filed on Sep. 11, 2023.

(51) Int. Cl. A61N 5/06

(52) U.S. Cl. CPC A61N 5/0619; A61N 2005/0626; A61N 2005/0642

(57) ABSTRACT

The present application provides medical uses of biophoton generators, which can generate bioenergy for promoting health and wellness. In particular, present application provides methods of needleless biophoton therapies for optimizing meridian energy, either alone or in combination with the use of another therapeutic energy source, by using biophoton generators.

### CROSS-REFERENCE TO RELATED APPLICATION

This application claims priority to U.S. Provisional Patent Application No. 63/581,755 filed on Sep. 11, 2023, the disclosure of which is incorporated herein by reference in its entirety.

### TECHNICAL FIELD

The present application provides medical uses of biophoton generators, which can generate bioenergy for promoting health and wellness. In particular, the present application provides methods of needleless biophoton therapies for optimizing meridian energy, either alone or in combination with the use of another therapeutic energy source.

### BACKGROUND OF THE APPLICATION

Maintaining the body free of any disease is a universal desire. Due to exposure to a variety of pathogenic factors, people inevitably have aged cells/organs and get certain diseases during their lifetime. It is acceptable if the disease can be postponed as long as possible, or occur at the very late stage of life when the important cells of the body lost their cellular activities. Preventing a disease from happening is the ideal medicine. If any therapy can directly increase the life force to enhance innate healing, that therapy would be safer and much preferred. One of them was the energy medicine using subtle energy of scalar wave.

Subtle energy research has helped advance the understanding of scalar waves. There are many different types of scalar waves, such as vital scalar waves (corresponding with the vital life force or “Qi” body), emotional scalar waves, mental scalar waves, and causal scalar waves. In essence, as far as we are aware, all “subtle” energies are made up of various types of scalar waves and biophoton. Scalar wave energy has also been safely used for many years as medical devices. A number of scalar wave energy devices are in the consumer market, such as Scalar Wave Laser, or Tesla watches, and have been used to treat a variety of diseases.

In addition, it is advantageous to have more than one energy source to treat certain disorders. For example, infrared (IR) treatment is a safe and effective therapeutic energy source, which has been proved by a lot of studies.

There remains a need for developing methods of using energy medicine for preventing and/or treating diseases and disorders.

The inventors of the present application have developed and disclosed biophoton generators (or called bioenergy generators), which are capable of broadcasting scalar waves in a circulatory manner so that the whole body can be uniformly energized from all directions. The present application describes a variety of methods of using such biophoton generators for medical benefits.

### BRIEF SUMMARY OF THE INVENTION

The present application related to methods of using biophoton generators for medical benefits, either alone or in combination with another therapeutic energy source.

In one general aspect, the present application provides a method of needleless biophoton therapy for optimizing energy of a meridian of a subject, the method comprising:
a) placing a biophoton generator close to the subject within a distance of no more than 20 meters;
b) keeping the biophoton generator close to the subject for at least 5 minutes per day for a period of time; and
c) observing the subject's improvement of the meridian for at least 1 day.

In some embodiments, the subject has a disease that is treatable with acupuncture.

In certain embodiments, the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

In some embodiments, the meridian is a string connecting acupuncture point, or a passageway through which energy flows throughout the body.

In some embodiments, the method uses at least one biophoton generator, preferably 2 to 20 biophoton generators, and more preferably, 4 to 14 biophoton generators.

In some embodiments, the biophoton generator is kept close to the subject for 5 minutes to 24 hours per day.

In some embodiments, the distance in step a) is from 1 cm to 20 meters, preferably from 0.2 meter to 0.5 meter.

In some embodiments, the period of time in step b) is from 1 day to 1 year, preferably from 1 week to 2 months.

In another general aspect, the present application provides a method of needleless biophoton therapy for optimizing energy of at least one meridian of a subject, the method comprising:
a) using at least one biophoton generator to energize the at least one meridian or to improve the function of the at least one meridian, for at least 5 minutes per day for a period of time; and
b) observing the efficacy of the method in improving the at least one meridian for at least 1 day.

In some embodiments, the subject has a disease that is treatable with acupuncture.

In certain embodiments, the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

In some embodiments, the meridian is a string connecting acupuncture point, or a passageway through which energy flows throughout the body.

In some embodiments, the method uses at least one biophoton generator, preferably 2 to 20 biophoton generators, and more preferably, 4 to 14 biophoton generators.

In some embodiments, the biophoton generator is kept close to the subject for 5 minutes to 24 hours per day.

In some embodiments, the distance in step a) is from 1 cm to 20 meters, preferably from 0.2 meter to 0.5 meter.

In some embodiments, the period of time in step b) is from 1 day to 1 year, preferably from 1 week to 2 months.

In another general aspect, the present application provides a method of combinational needleless biophoton therapy for optimizing energy of a meridian of a subject, the method comprising:
a) placing a biophoton generator close to the subject's head within a distance of no more than 20 meters, while placing another therapeutic energy source close to the subject;
b) keeping the biophoton generator and the another therapeutic energy source close to the subject for at least 5 minutes per day for a period of time; and
c) observing the subject's improvement of the meridian for at least 1 day.

In some embodiments, the subject has a disease that is treatable with acupuncture.

In certain embodiments, the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

In some embodiments, the meridian is a string connecting acupuncture point, or a passageway through which energy flows throughout the body.

In some embodiments, the method uses at least one biophoton generator and at least one another therapeutic energy source.

In some embodiments, the another therapeutic energy source generates infrared light or scalar wave energy.

In some embodiments, the another therapeutic energy source is acupuncture.

In some embodiments, the biophoton generator is kept close to the subject for 5 minutes to 24 hours per day.

In some embodiments, the distance in step a) is from 1 cm to 20 meters, preferably from 0.2 meter to 0.5 meter.

In some embodiments, the period of time in step b) is from 1 day to 1 year, preferably from 1 week to 2 months.

It is an object of the presently disclosed subject matter to provide methods of needleless biophoton therapy for optimizing energy of meridians by using a biophoton generator, either alone or in combination with another therapeutic energy source.

An object of the presently disclosed subject matter having been stated hereinabove, and which is achieved in whole or in part by the presently disclosed subject matter, other objects will become evident as the description proceeds when taken in connection with the accompanying drawings as described herein below.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments sown in the drawings.

FIG. 1 depicts changes of meridian energy after a patient with Parkinson's disease used biophoton for 2 weeks.

FIG. 2 depicts changes of meridian energy after a patient with traumatic brain injury used biophoton for 24 hours.

FIG. 3 depicts changes of meridian energy after another patient with traumatic brain injury used biophoton for 24 hours.

FIG. 4 depicts the energy of the meridian of “Yin of Lungs” of a patient with post-COVID conditions at the baseline.

FIG. 5 depicts the energy of the meridian of “Yin of Lungs” of a patient with post-COVID conditions using biophoton generators for 24 hours.

FIG. 6 depicts the energy of the meridian of “Yin of Spleen” of a patient with post-COVID conditions at the baseline.

FIG. 7 depicts the energy of the meridian of “Yin of Spleen” of a patient with post-COVID conditions using biophoton generators for 24 hours.

FIG. 8 depicts Bio-Well machine and one of the many report figures.

### DETAILED DESCRIPTION OF THE INVENTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

### Definitions

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a distance or a time range described herein, are to be understood as being modified in all instances by the term “about.” Thus, a numerical value typically includes ±10% of the recited value. For example, the recitation of “10 minutes” includes 9 minutes and 11 minutes. As used herein, the use of a numerical range expressly includes all possible subranges, all individual numerical values within that range, including integers within such ranges and fractions of the values unless the context clearly indicates otherwise.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

The terms of “biophoton generator, “bioenergy generator” and “life force generator” are interchangeable throughout the present application. The biophoton generators can be either stationary or portable, and can produce life force (Qi, or biophoton) and meet a variety of purposes. The biophoton generators are powerful in producing life force without using any electricity, either plug-in or battery. The bioenergy field is formed by the presence of the biophoton generator. The bioenergy field is around the entire body and increases the cellular bioenergy, to increase cellular repair rate, to decrease cell destruction rate, to promote the cell metabolism, to detoxify toxins or free radicals, to remove waste products from the cells, and to bio-recharge cells to the full voltage level for maintaining optimum health of a mammal. All cells and all organs in the whole body become energized.

The biophoton generator includes fine pieces of natural stone selected from the group of diamond, amethyst, tourmaline, jadestone with any color, and obsidian. It further includes fine pieces of natural sand selected from the group of white sand, black sand, pink sand, red sand, yellow sand, orange sand, and green sand. It further includes fine pieces of metal selected from the group of steel, iron, copper, zinc, aluminum, calcium, magnesium, silver, and gold. It further includes water and a supporting material operable to hold the natural sand, the natural stone, the metal, and the water together.

The biophoton generators and manufacturing process thereof are described in U.S. patent application Ser. No. 17/409,665, hereby incorporated by reference herein in its entirety.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

### Methods of Needleless Biophoton Therapy

In one general aspect, provided herein is a method of needleless biophoton therapy for optimizing energy of a meridian of a subject, the method comprising:
a) placing a biophoton generator close to the subject within a distance of no more than 20 meters;
b) keeping the biophoton generator close to the subject for at least 5 minutes per day for a period of time; and
c) observing the subject's improvement of the meridian for at least 1 day.

In some embodiments, the subject has a disease that is treatable with acupuncture.

As used herein, the terms “treat,” “treatable,” “treating,” and “treatment” are all intended to refer to an amelioration or reversal of at least one measurable physical parameter related to a disease, which is not necessarily discernible in the subject, but can be discernible in the subject. The terms “treat,” “treatable,” “treating,” and “treatment,” can also refer to causing regression, preventing the progression, or at least slowing down the progression of a disease or a disorder. In a particular embodiment, “treat,” “treatable,” “treating,” and “treatment” refer to an alleviation, prevention of the development or onset, or reduction in the duration of one or more symptoms associated with a disease or disorder. In a particular embodiment, “treat,” “treatable,” “treating,” and “treatment” refer to prevention of the recurrence of a disease or a disorder. In a particular embodiment, “treat,” “treatable,” “treating,” and “treatment” refer to an increase in the survival of a subject having a disease or a disorder. In a particular embodiment, “treat,” “treatable,” “treating,” and “treatment” refer to elimination of a disease or a disorder in a subject.

As used herein, acupuncture is a technique in which practitioners insert fine needles into the skin to treat health problems. The needles may be manipulated manually or stimulated with small electrical currents (electroacupuncture). Acupuncture has been in use in some form for at least 2,500 years with demonstrated therapeutic effects. It originated from traditional Chinese medicine (TCM) but has gained popularity worldwide since the 1970s. According to National Center for Complementary and Integrative Health, acupuncture is used in 103 of 129 countries, and in the United States, there is a 50 percent increase in the number of acupuncture users between 2002 and 2012.

According to embodiments of the present application, the methods described herein distinct from acupuncture therapy in that the methods do not require the use of needles, but may have the same functions as acupuncture therapy in terms of prevention and/or treatment of certain diseases/disorders. That is to say, the methods described in the present application can prevent and/or treat all the diseases/disorders that are treatable by acupuncture.

In certain embodiments, the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

As used herein, the term “meridian” originated from traditional Chinese medicine (TCM) theory and refers to a meridian network. The meridian network contains 12 standard meridians (also called Principal Meridians) where each meridian corresponds to either a hollow or solid organ, interacting with it and extending along a particular extremity (i.e. arm or leg), as well as 8 extraordinary channels, two of which have their own sets of points, and the remaining ones connecting points on other channels. The meridian network also contains 12 tendinomuscular meridians and 12 divergent meridians.

The 12 standard meridians are divided into Yin and Yang groups. The Yin meridians of the arm are the Lung, Heart, and Pericardium. The Yang meridians of the arm are the Large Intestine, Small Intestine, and Triple Burner. The Yin Meridians of the leg are the Spleen, Kidney, and Liver. The Yang meridians of the leg are Stomach, Bladder, and Gall Bladder.

In some embodiments, the meridians are strings connecting acupuncture points, which are considered as passageways through which energy flows throughout the body. Acupuncture treatments can improve the flow of energy through the meridian network. There are about 400 acupuncture points, most of which are situated along the major 20 pathways (i.e. 12 primary and 8 extraordinary channels).

In some embodiments, the method further comprises recording the subject's energy of the meridian during the period of time. The meridian energy can be measured by any method known in the field, which includes, but not limited to, biometer such as discharge visualization (GDV) technique (Bio-Well). Bio-Well has been developed by an international team led by Dr. Konstantin Korotkov and brings the powerful technology known as Gas Discharge Visualization (GDV) technique to market in a more accessible way than ever before. It allows to foresee the functional disorders and to take preventive actions before the illness takes place. Bio-Well Test Reports include: Energy assessment of body systems; Before & after analysis of energy scans; Personalized meditation music based off of your results; Energy & wellness analysis; Sympathetic & parasympathetic balance report; Chakra reports (Chakras symbolize energy centers and correspond to nerves, major organs, and areas of our spiritual body that impact our physical and emotional well-being); Meridian energy flow reports (Meridians are energetic highways in the human body that allow for the flow of energy to circulate throughout the body. Meridians exist in pairs and each has multiple acupuncture points along the pathway). See FIG. 8 for the Bio-Well machine and one of the many report figures.

In some embodiments, the method further comprises observing the energy change of the meridian during the period of time. The observation of the energy change of the meridian can be achieved by comparing the energy at any different time points during the period time, such as the starting point (i.e., baseline) and the ending point of the period time.

In some embodiments, the method uses at least one biophoton generator, preferably 2 to 20 biophoton generators, and more preferably, 4 to 14 biophoton generators. The biophoton generators used in the method can be any size, such as 8 Oz, 16 Oz, 32 Oz, 64 Oz and 1.5 Gallons.

In some embodiments, the biophoton generator is kept close to the subject for 5 minutes to 24 hours per day, such as 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours per day, or any period of time in between thereof. In preferred embodiments, the biophoton generator is kept close to the subject for at least 8 hours per day.

In some embodiments, the distance in step a) is from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

According to the embodiment of the present application, the biophoton generator can be placed at any place that is close to the subject at any direction of the 3-dimensional space of the biophoton generator, and can be used any time of the day, such as daytime or night time, whenever the subject is awake or asleep. The biophoton generator can be used continuously or intermittently.

In some embodiments, the period of time in step b) is from 1 day to 1 year, such as 1 day, two days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any period of time in between thereof. In preferred embodiments, the period time is from 1 week to 2 months.

In some embodiments, the method of needleless biophoton therapy is started during any stage of clinical treatment of the disease. In certain embodiments, the method is started immediately after the disease is diagnosed.

In some embodiments, the method of needleless biophoton therapy increases the energy of the meridian after the subject uses the biophoton generator for a period of time. In certain embodiments, the method of needleless biophoton therapy increase the energy of all 12 standard meridians. In certain embodiments, the method of needleless biophoton therapy increase the energy of majority of 12 standard meridians, such as 7, 8, 9, 10, or 11 meridians.

In some embodiments, the method of needleless biophoton therapy further improves the amount of energy in the organs (Energy Balance), the percent deviation of the energy measurements from the hands (Organ Disbalance), the level of anxiety (Stress), the energy re-distribution for different organs and systems based on area parameters (Area Diagram), the energy parameters of particular sectors of the body (Energy Diagram), and the energy of each of the 12 major meridians of the body (Energy Diagram).

In certain embodiments, the organ is cardiovascular system, heart, colon, pancreas, spleen, liver, pituitary gland, thyroid gland, adrenals, spine (cervical zone, thorax zone, or lumbar zone), sacrum, coccyx, pelvis minor zone, urogenital system, kidney, cars, nose, maxillary sinus, throat, larynx, trachea, cerebral zone (vessels or cortex), respiratory system, coronary vessels, thorax zone, epiphysis, hypothalamus, nervous system, immune system, right eye, left eye, jaw, teeth, or prostate.

In another general aspect, provided herein is a method of needleless biophoton therapy for optimizing energy of at least one meridian of a subject, the method comprising:
a) using at least one biophoton generator to energize the at least one meridian or to improve the function of the at least one meridian, for at least 5 minutes per day for a period of time; and
b) observing the efficacy of the method in improving the at least one meridian for at least 1 day.

In some embodiments, the subject has a disease that is treatable with acupuncture.

In certain embodiments, the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

In some embodiments, the meridians are strings connecting acupuncture points, which are considered as passageways through which energy flows throughout the body.

In some embodiments, the method further comprises recording the subject's progress in improving energy of the at least one meridian during the period of time. The meridian energy can be measured by any method known in the field, which includes, but not limited to, biometer such as discharge visualization (GDV) technique (Bio-Well).

In some embodiments, the method further comprises observing the efficacy of the method to determine an improvement of the at least one meridian during the period of time. The observation of the energy change of the meridian can be achieved by comparing the energy at any different time points during the period time, such as the starting point (i.e., baseline) and the ending point of the period time.

In some embodiments, the method uses at least one biophoton generator, preferably 2 to 20 biophoton generators, and more preferably, 4 to 14 biophoton generators. The biophoton generators used in the method can be any size, such as 8 Oz, 16 Oz, 32 Oz, 64 Oz and 1.5 Gallons.

In some embodiments, the biophoton generator is kept close to the subject for 5 minutes to 24 hours per day, such as 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours per day, or any period of time in between thereof. In preferred embodiments, the biophoton generator is kept close to the subject for at least 8 hours per day.

In some embodiments, the distance in step a) is from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

According to the embodiment of the present application, the biophoton generator can be placed at any place that is close to the subject at any direction of the 3-dimensional space of the biophoton generator, and can be used any time of the day, such as daytime or night time, whenever the subject is awake or asleep. The biophoton generator can be used continuously or intermittently.

In some embodiments, the period of time in step b) is from 1 day to 1 year, such as 1 day, two days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any period of time in between thereof. In preferred embodiments, the period time is from 1 week to 2 months.

In some embodiments, the method of needleless biophoton therapy is started during any stage of clinical treatment of the disease. In certain embodiments, the method is started immediately after the disease is diagnosed.

In some embodiments, the method of needleless biophoton therapy increases the energy of the meridian after the subject uses the biophoton generator for a period of time. In certain embodiments, the method of needleless biophoton therapy increase the energy of all 12 standard meridians. In certain embodiments, the method of needleless biophoton therapy increase the energy of majority of 12 standard meridians, such as 7, 8, 9, 10, or 11 meridians.

In some embodiments, the method of needleless biophoton therapy further improves the amount of energy in the organs (Energy Balance), the percent deviation of the energy measurements from the hands (Organ Disbalance), the level of anxiety (Stress), the energy re-distribution for different organs and systems based on area parameters (Area Diagram), the energy parameters of particular sectors of the body (Energy Diagram), and the energy of each of the 12 major meridians of the body (Energy Diagram).

In certain embodiments, the organ is cardiovascular system, heart, colon, pancreas, spleen, liver, pituitary gland, thyroid gland, adrenals, spine (cervical zone, thorax zone, or lumbar zone), sacrum, coccyx, pelvis minor zone, urogenital system, kidney, cars, nose, maxillary sinus, throat, larynx, trachea, cerebral zone (vessels or cortex), respiratory system, coronary vessels, thorax zone, epiphysis, hypothalamus, nervous system, immune system, right eye, left eye, jaw, teeth, or prostate.

In another general aspect, provided herein is a method of combinational needleless biophoton therapy for optimizing energy of a meridian of a subject, the method comprising:
a) placing a biophoton generator close to the subject's head within a distance of no more than 20 meters, while placing another therapeutic energy source close to the subject;
b) keeping the biophoton generator and the another therapeutic energy source close to the subject for at least 5 minutes per day for a period of time; and
c) observing the subject's improvement of the meridian for at least 1 day.

In some embodiments, the subject has a disease that is treatable with acupuncture.

In certain embodiments, the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

In some embodiments, the meridians are strings connecting acupuncture points, which are considered as passageways through which energy flows throughout the body.

In some embodiments, the method further comprises recording the subject's progress in improving energy of the meridian during the period of time. The meridian energy can be measured by any method known in the field, which includes, but not limited to, biometer such as discharge visualization (GDV) technique (Bio-Well).

In some embodiments, the method further comprises observing the efficacy of the method to determine an improvement of the meridian during the period of time. The observation of the energy change of the meridian can be achieved by comparing the energy at any different time points during the period time, such as the starting point (i.e., baseline) and the ending point of the period time.

In some embodiments, the method uses at least one biophoton generator and at least one another therapeutic energy source.

As used herein, the therapeutic energy source refers to any devices or methods that can generate energy for therapeutic purpose. The generated energy can be bioenergy, such as infrared light (IR) and scalar wave energy.

In certain embodiments, the another therapeutic energy source is acupuncture.

In certain embodiments, the another therapeutic energy source generates infrared light (IR) or scalar wave energy or other form of bioenergy.

In some embodiments, the method uses at least one biophoton generator, preferably 2 to 20 biophoton generators, and more preferably, 4 to 14 biophoton generators. The biophoton generators used in the method can be any size, such as 8 Oz, 16 Oz, 32 Oz, 64 Oz and 1.5 Gallons.

In some embodiments, the combinational needleless biophoton therapy is applied for 5 minutes to 24 hours per day, such as 5 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours per day, or any period of time in between thereof. In preferred embodiments, the combinational needleless biophoton therapy is applied for at least 8 hours per day.

In some embodiments, the distance in step a) is from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

In some embodiments, the another therapeutic energy source is placed close the subject with a distance of from 1 cm to 20 meters, such as 1 cm, 10 cm, 50 cm, 100 cm, 2 meters, 3 meters, 4 meters, 5 meters, 6 meters, 7 meters, 8 meters, 9 meters, 10 meters, 11 meters, 12 meters, 13 meters, 14 meters, 15 meters, 16 meters, 17 meters, 18 meters, 19 meters, or 20 meters, or any distance in between thereof. In preferred embodiments, the distance is from 0.2 meter to 0.5 meter.

According to the embodiment of the present application, the combinational needleless biophoton therapy can be placed at any place that is close to the subject at any direction of the 3-dimensional space of the biophoton generator, and can be applied any time of the day such as daytime or night time, whenever the subject is awake or asleep. The combinational needleless biophoton therapy can be applied continuously or intermittently.

In some embodiments, the period of time in step b) is from 1 day to 1 year, such as 1 day, two days, 3 days, 4 days, 5 days, 6 days, 1 week, 2 weeks, 3 weeks, 4 weeks, 1 month, 2 months, 3 months, 4 months, 5 months, 6 months, 7 months, 8 months, 9 months, 10 months, 11 months, 1 year, or any period of time in between thereof. In preferred embodiments, the period time is from 1 week to 2 months.

In some embodiments, the combinational needleless biophoton therapy is started during any stage of clinical treatment of the disease. In certain embodiments, the combinational needleless biophoton therapy is started immediately after the disease is diagnosed.

In some embodiments, the combinational needleless biophoton therapy increases the energy of the meridian after the subject uses the biophoton generator for a period of time. In certain embodiments, the combinational needleless biophoton therapy increase the energy of all 12 standard meridians. In certain embodiments, the method of needleless biophoton therapy increase the energy of majority of 12 standard meridians, such as 7, 8, 9, 10, or 11 meridians.

In some embodiments, the combinational needleless biophoton therapy further improves the amount of energy in the organs (Energy Balance), the percent deviation of the energy measurements from the hands (Organ Disbalance), the level of anxiety (Stress), the energy re-distribution for different organs and systems based on area parameters (Area Diagram), the energy parameters of particular sectors of the body (Energy Diagram), and the energy of each of the 12 major meridians of the body (Energy Diagram).

In certain embodiments, the organ is cardiovascular system, heart, colon, pancreas, spleen, liver, pituitary gland, thyroid gland, adrenals, spine (cervical zone, thorax zone, or lumbar zone), sacrum, coccyx, pelvis minor zone, urogenital system, kidney, cars, nose, maxillary sinus, throat, larynx, trachea, cerebral zone (vessels or cortex), respiratory system, coronary vessels, thorax zone, epiphysis, hypothalamus, nervous system, immune system, right eye, left eye, jaw, teeth, or prostate.

### EXAMPLES

The following examples are provided to illustrate the uses of bioenergy generator and to aid those of ordinary skill in the art in practicing the invention. These examples are not intended to limit the scope of the invention.

### Instruments

The gas discharge visualization (GDV) technique (Bio-Well) was used to measure bioenergy of the human body. The Bio-Well device was developed in Russia in 1995 by an international team led by Dr. Konstantin Korotkov, a visionary biophysicist known for bringing the gas discharge visualization (GDV) technique to market in the form of the Bio-Well camera. His device has made the GDV method of scientific discovery more accessible to the world than ever before.

The GDV technique identifies the functional psychoemotional and physiological state of a person using their fingertips. The technology especially lends itself to the emerging field of medical biometrics, wherein biomedical patterns derived from unique features, such as fingerprints, faces, iris, and hand geometry, are studied to learn how these patterns are associated with various diseases.

Rather unassuming in appearance, the Bio-Well consists of a desktop camera with accompanying Cloud software that allows an examiner to quickly and easily conduct human energy scans by capturing finger glows from the pads of each finger on each hand. Specifically, the Bio-Well device can assess the overall energy level of the organism (Energy, joules×10⁻²), the amount of energy in the organs (Energy Balance), the percent deviation of the energy measurements from the hands (Organ Disbalance), the level of anxiety (Stress), the energy re-distribution for different organs and systems based on area parameters (Area Diagram), the energy parameters of particular sectors of the body (Energy Diagram, joules×10⁻²), and the energy of each of the 12 major meridians of the body (Energy Diagram, joules×10⁻²). Below are the energy changes of the meridians of the patients with special health conditions.

### Example 1. Changes of Meridian Energy after a Patient with Parkinson's Disease Used Biophoton for 2 Weeks

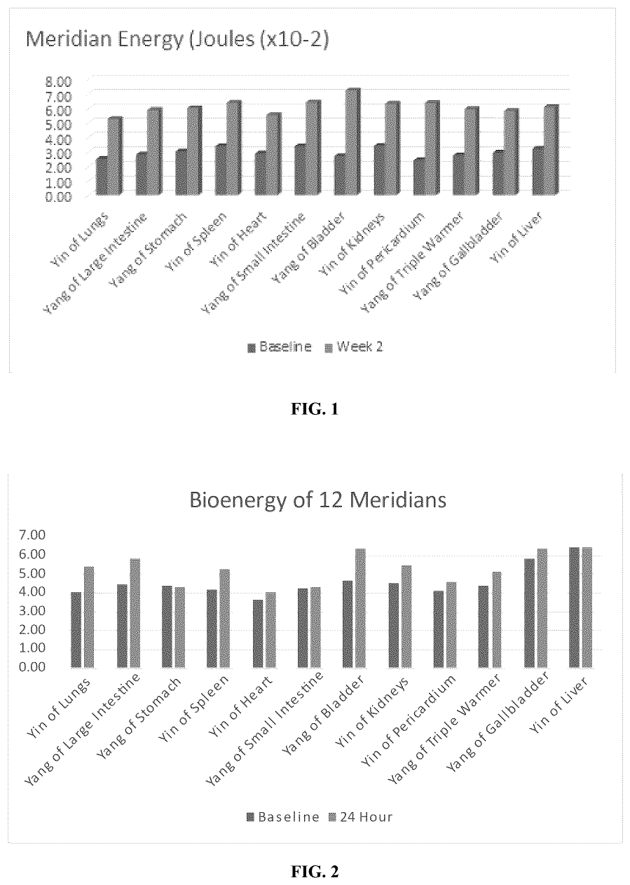

One patient with Parkinson's disease slept on a Tesla MedBed that was a regular hotel bed, under which 14 biophoton generators (each has a size of 32 oz) were placed. The patient used the MedBed for at least 8 hours during overnight sleep, and used it whenever he wanted during daytime. His body was within 0.5 meters from the biophoton generators. The meridian energy was measured at baseline (before using the MedBed) and at 2 weeks after he started to use the MedBed. His regular treatment with any prescription or OTC drugs was not alternated from the baseline to the end of the study. The meridian energy change was shown in FIG. 1 and Table 1 below.

TABLE 1

| Meridian | Bioenergy | Baseline | Week 2 |
|---|---|---|---|
| Yin of Lungs | Joules (×10⁻²) | 2.50 | 5.48 |
| Yang of Large | Joules (×10⁻²) | 2.80 | 5.52 |
| Intestine | | | |
| Yang of Stomach | Joules (×10⁻²) | 3.01 | 5.80 |
| Yin of Spleen | Joules (×10⁻²) | 3.37 | 6.95 |
| Yin of Heart | Joules (×10⁻²) | 2.87 | 5.37 |
| Yang of Small | Joules (×10⁻²) | 3.36 | 5.73 |
| Intestine | | | |
| Yang of Bladder | Joules (×10⁻²) | 2.68 | 4.97 |
| Yin of Kidneys | Joules (×10⁻²) | 3.39 | 6.61 |
| Yin of Pericardium | Joules (×10⁻²) | 2.41 | 5.55 |
| Yang of Triple | Joules (×10⁻²) | 2.75 | 5.70 |
| Warmer | | | |
| Yang of Gallbladder | Joules (×10⁻²) | 2.92 | 6.19 |
| Yin of Liver | Joules (×10⁻²) | 3.18 | 6.88 |

The above results clearly demonstrated that, after using biophoton generators for 2 weeks, the patient with Parkinson's Disease increased the energy level for each of all 12 meridians.

### Example 2. Changes of Meridian Energy after Another Patient with Parkinson's Disease Used Biophoton for 2 Weeks

Another patient with Parkinson's disease slept on a Tesla MedBed that was a regular hotel bed, under which 14 biophoton generators (each has a size of 32 oz) were placed. The patient used the MedBed at least for 8 hours during overnight sleep, and used the it whenever he wanted during daytime. His body was within 0.5 meters from the biophoton generators. The meridian energy was measured at baseline (before using the MedBed) and 14 days after he started to use the MedBed. The patient's regular treatment with any prescription or OTC drugs was not alternated from the baseline to the end of the study. The meridian energy change was shown in Table 2 below.

TABLE 2

| Meridian | Bioenergy | Baseline | Week 2 |
|---|---|---|---|
| Yin of Lungs | Joules (×10⁻²) | 3.46 | 6.47 |
| Yang of Large | Joules (×10⁻²) | 3.15 | 5.66 |
| Intestine | | | |
| Yang of Stomach | Joules (×10⁻²) | 3.83 | 4.97 |
| Yin of Spleen | Joules (×10⁻²) | 4.57 | 4.43 |
| Yin of Heart | Joules (×10⁻²) | 3.76 | 4.42 |
| Yang of Small | Joules (×10⁻²) | 4.56 | 5.50 |
| Intestine | | | |
| Yang of Bladder | Joules (×10⁻²) | 5.18 | 5.90 |
| Yin of Kidneys | Joules (×10⁻²) | 3.79 | 5.58 |
| Yin of Pericardium | Joules (×10⁻²) | 2.40 | 4.42 |
| Yang of Triple | Joules (×10⁻²) | 4.26 | 4.57 |
| Warmer | | | |
| Yang of Gallbladder | Joules (×10⁻²) | 2.74 | 4.54 |
| Yin of Liver | Joules (×10⁻²) | 4.55 | 6.34 |

The above results clearly demonstrated that, after using biophoton generators for 2 weeks, the patient with Parkinson's Disease increased the energy level for each of all 12 meridians.

### Example 3. Changes of Meridian Energy after a Patient with Chronic Stroke Used Biophoton for 2 Weeks

The patient had chronic stroke for more than 6 months and was declared as a permanent disability. The patient slept on a Tesla MedBed that was a regular hotel bed, under which 14 biophoton generators (each has a size of 32 oz) were placed. The patient used the MedBed at least for 8 hours during overnight sleep, and used the it whenever he wanted during daytime. His body was within 0.5 meters from the biophoton generators. The meridian energy was measured at baseline (before using the MedBed) and 14 days after he started to use the MedBed. The patient's regular treatment with any prescription or OTC drugs was not alternated from the baseline to the end of the study. After the patient used 14 biophoton generators for 2 weeks, the mobility and overall life quality was improved significantly. The meridian energy change was shown in Table 3 below.

TABLE 3

| Meridian | Bioenergy | Baseline | Week 2 |
|---|---|---|---|
| Yin of Lungs | Joules (×10⁻²) | 4.31 | 4.70 |
| Yang of Large | Joules (×10⁻²) | 4.16 | 5.06 |
| Intestine | | | |
| Yang of Stomach | Joules (×10⁻²) | 3.82 | 3.99 |
| Yin of Spleen | Joules (×10⁻²) | 4.33 | 3.91 |
| Yin of Heart | Joules (×10⁻²) | 3.77 | 4.09 |
| Yang of Small | Joules (×10⁻²) | 3.69 | 4.29 |
| Intestine | | | |
| Yang of Bladder | Joules (×10⁻²) | 5.24 | 5.97 |
| Yin of Kidneys | Joules (×10⁻²) | 4.84 | 5.22 |
| Yin of Pericardium | Joules (×10⁻²) | 3.82 | 4.25 |
| Yang of Triple | Joules (×10⁻²) | 4.18 | 4.12 |
| Warmer | | | |
| Yang of Gallbladder | Joules (×10⁻²) | 4.72 | 4.46 |
| Yin of Liver | Joules (×10⁻²) | 5.47 | 5.57 |

This above results demonstrated that, after using biophoton generators for 2 weeks, the patient with chronic stroke increased the energy level for the majority of the 12 meridians.

### Example 4. Changes of Meridian Energy after a Patient with Traumatic Brain Injury Used Biophoton for 24 Hours

The patient slept on a Tesla MedBed that was a regular hotel bed, under which 14 biophoton generators (each has a size of 32 oz) were placed. The patient used the MedBed at least for 8 hours during overnight sleep, and used the it whenever he wanted during daytime. His body was within 0.5 meters from the biophoton generators. The patient's regular treatment with any prescription or OTC drugs was not alternated from the baseline to the end of the study. The meridian energy was measured at baseline (before using the MedBed) and 24 hours after he started to use the MedBed. The meridian energy change was shown in FIG. 2, and the results demonstrated that, after using biophoton generators for 24 hours, the patient with traumatic brain injury increased the energy level for the majority of the 12 meridians.

### Example 5. Changes of Meridian Energy after Another Patient with Traumatic Brain Injury Used Biophoton for 24 Hours

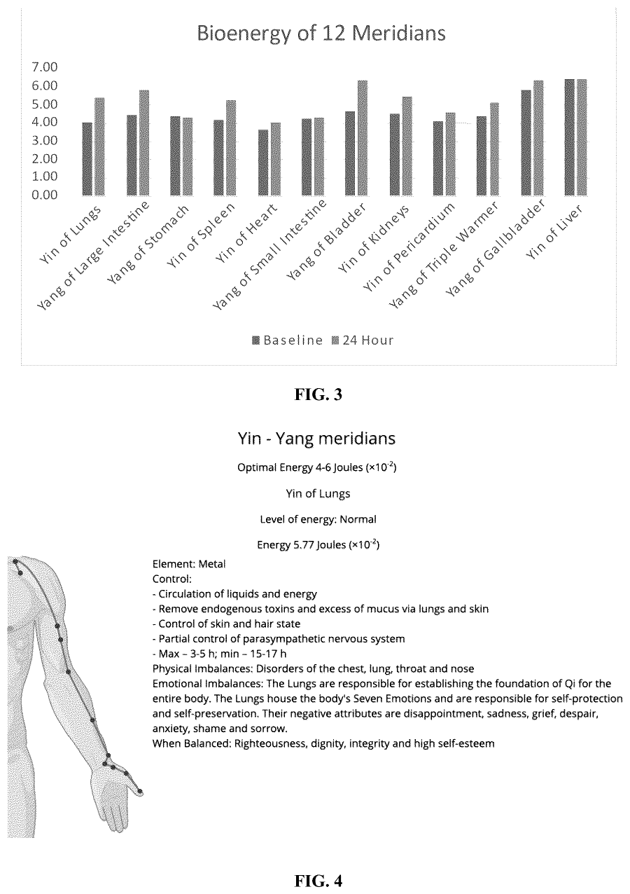

The patient slept on a Tesla MedBed that was a regular hotel bed, under which 14 biophoton generators (each has a size of 32 oz) were placed. The patient used the MedBed at least for 8 hours during overnight sleep, and used the it whenever he wanted during daytime. His body was within 0.5 meters from the biophoton generators. The patient's regular treatment with any prescription or OTC drugs was not alternated from the baseline to the end of the study. The meridian energy was measured at baseline (before using the MedBed) and 24 hours after he started to use the MedBed. The meridian energy change was shown in FIG. 3, and the results demonstrated that, after using biophoton generators for 24 hours, the patient with traumatic brain injury increased the energy level for the majority of the 12 meridians.

### Example 6. Changes of Meridian Energy after a Patient with Alzheimer's Disease Used Biophoton for 4 Weeks

The patient slept on a Tesla MedBed that was a regular hotel bed, under which 14 biophoton generators (each has a size of 32 oz) were placed. The patient used the MedBed at least for 8 hours during the overnight sleep, and used the it whenever he wanted during daytime. His body was within 0.5 meters from the biophoton generators. The patient's regular treatment with any prescription or OTC drugs were not alternated from the baseline to the end of the study. The meridian energy was measured at baseline (before using the MedBed) and 4 weeks after he started to use the MedBed. The meridian energy change was shown in Table 4 below.

TABLE 4

| Baseline | Bioenergy | Baseline | Week 4 |
|---|---|---|---|
| Yin of Lungs | Joules (×10⁻²) | 6.10 | 6.55 |
| Yang of Large Intestine | Joules (×10⁻²) | 5.54 | 5.06 |
| Yang of Stomach | Joules (×10⁻²) | 4.01 | 6.49 |
| Yin of Spleen | Joules (×10⁻²) | 4.94 | 6.26 |
| Yin of Heart | Joules (×10⁻²) | 4.23 | 4.33 |
| Yang of Small Intestine | Joules (×10⁻²) | 4.20 | 4.81 |
| Yang of Bladder | Joules (×10⁻²) | 6.52 | 6.47 |
| Yin of Kidneys | Joules (×10⁻²) | 6.37 | 6.74 |
| Yin of Pericardium | Joules (×10⁻²) | 4.68 | 5.20 |
| Yang of Triple Warmer | Joules (×10⁻²) | 5.05 | 5.83 |
| Yang of Gallbladder | Joules (×10⁻²) | 5.50 | 5.67 |
| Yin of Liver | Joules (×10⁻²) | 7.36 | 8.72 |

The above results demonstrated that, after using biophoton generators for 4 weeks, the patient with Alzheimer's disease increased the energy level for the majority of the 12 meridians.

### Example 7. Changes of Meridian Energy after a Patient with Post-COVID Conditions Used Biophoton for 24 Hours

The patient slept on a Tesla MedBed that was a regular hotel bed, under which 14 biophoton generators (each has a size of 32 oz) were placed. The patient used the MedBed at least for 8 hours during overnight sleep, and used the it whenever he wanted during daytime. His body was within 0.5 meters from the biophoton generators. The patient's regular treatment with any prescription or OTC drugs was not alternated from the baseline to the end of the study. The meridian energy was measured at baseline (before using the MedBed) and 24 hours after he started to use the MedBed.

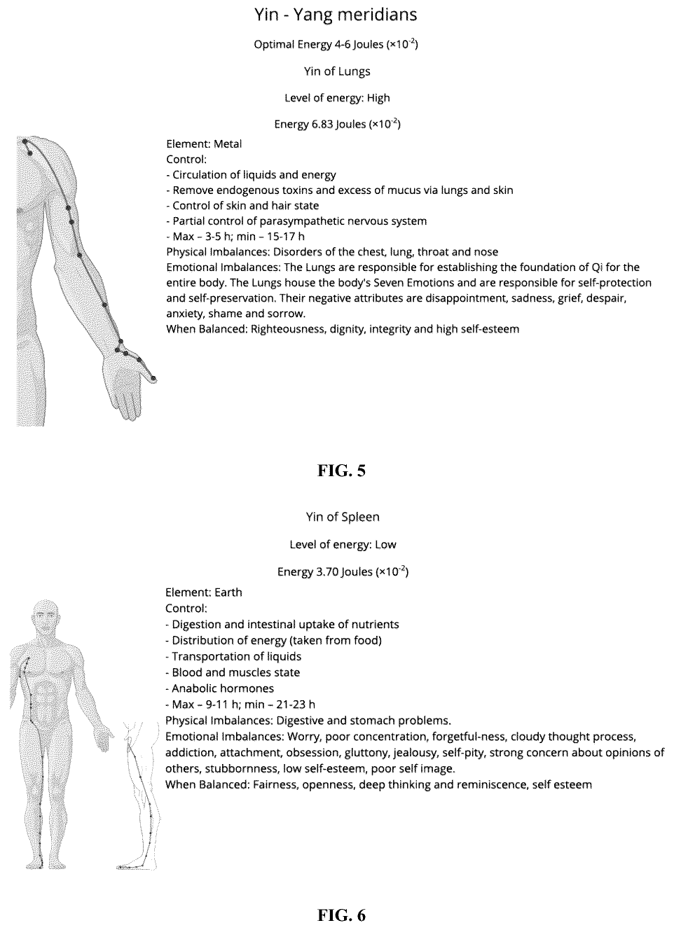

With respect to the energy of the meridian of “Yin of Lungs,” it was 5.77 Joules (×10⁻²) at the baseline (see FIG. 4); then, it increased to 6.83 Joules (×10⁻²) after using biophoton generators for 24 hours (see FIG. 5).

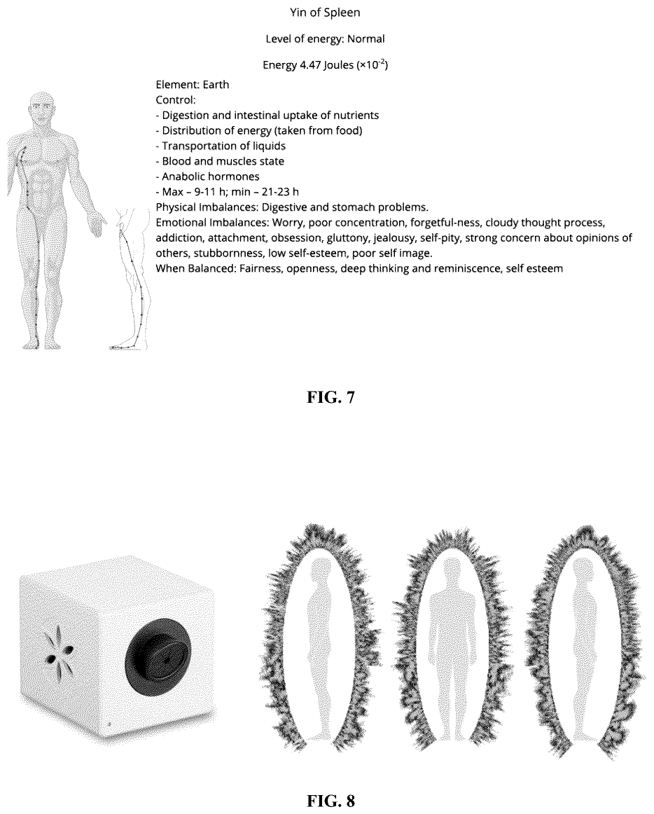

With respect to the energy of the meridian of “Yin of Spleen,” it was 3.70 Joules (×10⁻²) at the baseline (see FIG. 6); then, it increased to 4.47 Joules (×10⁻²) after using biophoton generators for 24 hours (see FIG. 7).

In addition, the above changes were well correlated to the recovery of the patient's overall health, increased breathing capacity and reduced pain.

### Example 8. Overall Energy Level Changes as Impacted by Using Biophoton Generators

The energy (Joules×10⁻²) was measured by the Bio-Well device for 22 patients after using the biophoton generators. Each patient slept on a Tesla MedBed that was a regular hotel bed, under which 8 biophoton generators (each has a size of 1.5-gallon) were placed. The distance between the user's body and the biophoton generator was less than 0.5 meter. The patients' regular treatments with any prescription or OTC drugs were not alternated from the baseline to the end of the study. As shown in Table 5 below, the average increase of 8.83 (Joules ×10⁻²) signifies the bioavailability of the photons generated by the biophoton generators after an approximate exposure time of one hour. Furthermore, the stress index lessened.

In summary, there were significant improvements in every parameter for the participants except for the chakras (%). It is noted that the chakras are related to the internal and external energy flow, and it is essential to note that they may change from one minute to the next.

TABLE 5
Energy Field, Energy Balance, Organ Imbalance,
and Stress Level of 22 Subjects

| | Before 1-hr | After 1-hr | Mean | |
|---|---|---|---|---|
| Indicator | Session | Session | Change | P Value |
| Energy Area Diagram Left | 0.287 | 0.587 | 0.300 | 0.00032 |
| Energy Area Diagram Right | 0.183 | 0.576 | 0.394 | 0.00008 |
| Energy Diagram | 0.703 | 0.878 | 0.175 | 0.00297 |
| Stress | 3.953 | 3.153 | −0.801 | 0.00254 |
| Energy (Joules ×10⁻²) | 49.438 | 58.268 | 8.830 | 0.00997 |
| Balance (%) | 93.156 | 96.189 | 3.034 | 0.03224 |
| Organ Disbalance | 7.833 | 4.508 | −2.669 | 0.01534 |
| Chakras (%) | 0.912 | 0.880 | 0.011 | 0.32046 |

The Bio-Well device allowed the examiner to analyze the energetic impact of the biophoton generators on the organs of the adult human body by combining and averaging the measurements of the 22 participants of the study.

The detailed rendering of the energy levels within the organs of one study participant from among the 22 was measured by the Bio-Well device before and then approximately 1.5 hours after using the generator, as shown in Table 6 below. When compared to the baseline, the energy level in each organ increased within either the left or right hemisphere of the brain after using the generator. Imbalances in the energy levels between the two hemispheres of the brain may signal physical or psychological distress and may lead to the early prediction of disease and/or psychological conditions.

TABLE 6
Energy Balance and Strength of the Organs

| | | | | Right- | Right- | |
|---|---|---|---|---|---|---|
| | | Left-Hand | | Hand | Hand | |
| | Left-Hand | Measured | | Measured | Measured | |
| | Measured | Energy | | Energy | Energy | |
| | Energy | Levels at 1 | | Levels at | Levels 1- | |
| | Levels at | Hour Use | Change | Baseline | Hour Use | Change |
| | Baseline | Energy, | in | Energy, | Energy, | in |
| | Energy, | Joules | Energy | joules | Joules | Energy |
| Organ | Joules (×10⁻²) | (×10⁻²) | (%) | (×10⁻²) | (×10⁻²) | (%) |
| 1. Cardiovascular | 3.42 | 3.57 | 4% | 3.64 | 4.30 | 18% |
| System | | | | | | |
| 2. Heart | 2.43 | 3.28 | 35% | 4.00 | 3.05 | −24% |
| 3. Colon - | 2.28 | 3.19 | 40% | 2.60 | 3.25 | 25% |
| transverse | | | | | | |
| 4. Pancreas, Spleen | 2.75 | 3.85 | 40% | 3.50 | 4.54 | 28% |
| 5. Liver | 4.46 | 5.76 | 29% | 4.88 | 5.11 | 8% |
| 6. Pituitary Gland | 3.51 | 4.01 | 14% | 2.70 | 3.70 | 37% |
| 7. Thyroid gland | 3.92 | 4.53 | 16% | 4.36 | 5.13 | 18% |
| 8. Adrenals | 3.40 | 4.26 | 25% | 3.46 | 5.50 | 59% |
| 9. Spine - cervical | 2.93 | 3.27 | 12% | 3.01 | 3.38 | 12% |
| zone | | | | | | |
| 10. Spine - thorax | 2.70 | 3.11 | 15% | 2.37 | 2.00 | −16% |
| zone | | | | | | |
| 11. Spine - lumbar | 3.32 | 3.92 | 18% | 2.93 | 3.12 | 7% |
| zone | | | | | | |
| 12. Sacrum | 4.21 | 5.42 | 29% | 5.25 | 6.16 | 17% |
| 13. Coccyx, Pelvis | 5.12 | 5.15 | 1% | 5.76 | 6.50 | 13% |
| minor zone | | | | | | |
| 14. Urogenital | 4.13 | 4.61 | 12% | 5.66 | 5.80 | 3% |
| system | | | | | | |
| 15. Kidney | 3.80 | 5.74 | 51% | 4.11 | 5.17 | 26% |
| 16. Ears, nose, | 2.58 | 2.38 | −8% | 3.05 | 3.60 | 18% |
| maxillary sinus | | | | | | |
| 17. Throat, larynx, | 3.92 | 4.37 | 12% | 4.51 | 5.18 | 15% |
| trachea | | | | | | |
| 18. Cerebral zone | 1.84 | 2.90 | 58% | 2.12 | 2.97 | 40% |
| (vessels) | | | | | | |
| 19. Respiratory | 4.00 | 5.38 | 35% | 5.17 | 4.99 | −4% |
| system | | | | | | |
| 20. Coronary vessels | 1.92 | 3.61 | 88% | 2.95 | 4.25 | 44% |
| 21. Thorax zone | 2.74 | 3.39 | 24% | 3.01 | 4.00 | 22% |
| 22. Epiphysis | 1.61 | 3.51 | 118% | 2.51 | 3.51 | 40% |
| 23. Hypothalamus | 2.43 | 3.93 | 62% | 2.43 | 3.94 | 62% |
| 24. Nervous system | 3.35 | 3.40 | 2% | 2.09 | 4.68 | 124% |
| 25. Immune system | 3.02 | 2.96 | −2% | 3.22 | 2.83 | −12% |
| 26. Spleen, Pancreas | 3.46 | 3.68 | 6% | 3.75 | 4.61 | 23% |
| 27. Right eye | 2.64 | 2.45 | −7% | 2.87 | 3.02 | 5% |
| 28. Left eye | 3.06 | 2.18 | −29% | 2.88 | 3.50 | 22% |
| 29. Jaw, Teeth | 3.46 | 2.84 | −18% | 2.27 | 4.01 | 76% |
| 30. Cerebral zone | 2.00 | 2.19 | 10% | 2.88 | 2.80 | −3% |
| (cortex) | | | | | | |
| 31. Eyes | 2.82 | 2.30 | −18% | 2.87 | 3.28 | 14% |
| 32. Prostate | 5.15 | 5.25 | 2% | 5.82 | 6.38 | 10% |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method of needleless biophoton therapy for optimizing energy of a meridian of a subject, the method comprising:
a) placing a biophoton generator close to the subject within a distance of no more than 20 meters;
b) keeping the biophoton generator close to the subject for at least 5 minutes per day for a period of time; and
c) observing the subject's improvement of the meridian for at least 1 day.

2. The method of claim 1, wherein the subject has a disease that is treatable with acupuncture.

3. The method of claim 2, wherein the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

4. The method of claim 1, wherein the meridian is a string connecting acupuncture point, or a passageway through which energy flows throughout the body.

5. The method of claim 1, further comprising recording the subject's energy of the meridian during the period of time.

6. The method of claim 1, further comprising observing the energy change of the meridian during the period of time.

7. The method of claim 1, wherein the method uses at least one biophoton generator.

8. The method of claim 1, wherein the biophoton generator is kept close to the subject for 5 minutes to 24 hours per day.

9. The method of claim 1, wherein the biophoton generator is placed at any place that is close to the subject.

10. The method of claim 1, wherein the method is started at any time of a day.

11. The method of claim 1, wherein the distance in step a) is from 1 cm to 20 meters, preferably from 0.2 meter to 0.5 meter.

12. The method of claim 1, wherein the period of time in step b) is from 1 day to 1 year, preferably from 1 week to 2 months.

13. The method of claim 2, wherein the method is started during any stage of clinical treatment of the disease.

14. The method of claim 13, wherein the method is started immediately after the disease is diagnosed.

15. A method of needleless biophoton therapy for optimizing energy of at least one meridian of a subject, the method comprising:
a) using at least one biophoton generator to energize the at least one meridian or to improve the function of the at least one meridian, for at least 5 minutes per day for a period of time; and
b) observing the efficacy of the method in improving the at least one meridian for at least 1 day.

16. The method of claim 15, wherein the subject has a disease that is treatable with acupuncture.

17. The method of claim 16, wherein the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

18-27. (canceled)

28. A method of combinational needleless biophoton therapy for optimizing energy of a meridian of a subject, the method comprising:
a) placing a biophoton generator close to the subject's head within a distance of no more than 20 meters, while placing another therapeutic energy source close to the subject;
b) keeping the biophoton generator and the another therapeutic energy source close to the subject for at least 5 minutes per day for a period of time; and
c) observing the subject's improvement of the meridian for at least 1 day.

29. The method of claim 28, wherein the subject has a disease that is treatable with acupuncture.

30. The method of claim 29, wherein the disease is selected from the group consisting of acute or chronic pain, severe pain, chemotherapy-induced and postoperative nausea and vomiting, dental pain, fibromyalgia, headaches, including tension headaches and migraines, labor pain, lower back pain, neck pain, osteoarthritis, menstrual cramps, respiratory disorders, allergic rhinitis, and tennis elbow.

31-40. (canceled)
